(19) United States
(12) Patent Application Publication
(10) Pub. No.: US 2005/0131349 A1
(43) Pub. Date: Jun. 16, 2005

(54) Shielded septum trocar seal

(75) Inventors: Jeremy J. Albrecht, Ladera Rancho, CA (US); Gary M. Johnson, Mission Viejo, CA (US); Henry Kahle, Trabuco Canyon, CA (US)

Correspondence Address: APPLIED MEDICAL RESOUCES CORPORATION, 22872 Avenida Empresa, Rancho Santa Margarita, CA, 92688, US

(73) Assignee: Applied Medical Resources Corporation

(21) Appl. No.: 11/000,123

(22) Filed: Nov. 30, 2004

Related U.S. Application Data
(60) Provisional application No. 60/529,455, filed on Dec. 12, 2003.

(51) Int. Cl. A61M 5/178

(52) U.S. Cl. 604/167.06

(57) ABSTRACT

The invention is directed to a trocar assembly having a channel defined along an elongate axis, the trocar assembly being adapted to receive a surgical instrument, the trocar assembly comprising a septum seal disposed in the channel including a seal tip having a proximal facing surface, the seal tip including portions defining an orifice; and a septum shield including a tubular member having a proximal end and a distal end, and a plurality of blades or leaflets protruding distally from the distal end of the tubular member, the septum shield being placed inside the septum seal such that the blades engage the proximal facing surface of the seal tip. The trocar assembly may further comprise a zero closure seal such as a double duckbill valve disposed in the channel outside of the septum seal. The septum shield operates to reduce the drag force and to minimize axial movement of the septum shield and the instrument during insertion and removal of the instrument through the septum seal. The septum shield may be formed from a rigid plastic material, and the septum seal may be formed of an elastomeric material. The blades or leaflets may overlap or offset one another. The orifice may be expandable to accommodate the instrument having a diameter of about 5-15 mm. The blades or leaflets have distal tips that glide or roll against the instrument during placement of the instrument. The distal tips may comprise of a combination of material, durometer and shield geometry to control the behavior of the septum shield. The trocar assembly may further comprise a second septum shield disposed outside of the septum seal.

This is a non-provisional application claiming the priority of provisional application Ser. No. 60/529,455, filed on Dec. 12, 2003, entitled “Shielded Septum Trocar Seal,” which is fully incorporated herein by reference.

### BACKGROUND OF THE INVENTION

1. Field of the Invention

This invention generally relates to medical access devices and, more specifically, to a shielded septum trocar seal.

2. Discussion of the Prior Art

Mechanical trocars typically include a cannula defining a working channel and a housing which encloses valves that function to inhibit the escape of insufflation gasses. The cannula of the trocar is adapted to be positioned across the abdominal wall of a patient using an obturator which is initially inserted into the working channel and then removed once the cannula is in place. Various elongated instruments can be inserted through the working channel of the trocar to reach and perform operative functions at a site within the abdomen. It is the function of the valves to engage the outer surface of such an instrument and form seals around the instrument to prevent the escape of insufflation gases.

Trocar valves are commonly formed from elastomeric materials which are highly susceptible to puncture and tearing by sharp instrument configurations. Since many instruments typically have sharp distal tips, it has become particularly desirable to protect the valves from these objects. More specifically, it is desirable to provide a mechanism that can protect the septum seal during the insertion of surgical instruments, to reduce the drag force encountered when placing or removing instruments through the seal, and to restrict septum seal movement in the axial direction of the seal and surgical instruments.

### SUMMARY OF THE INVENTION

In one embodiment of the invention, there is disclosed a trocar assembly having a channel defined along an elongate axis, the trocar assembly being adapted to receive a surgical instrument, the trocar assembly comprising a septum seal disposed in the channel including a seal tip having a proximal facing surface, the seal tip including portions defining an orifice; and a septum shield including a tubular member having a proximal end and a distal end, and a plurality of blades or leaflets protruding distally from the distal end of the tubular member, the septum shield being placed inside the septum seal such that the blades or leaflets engage the proximal facing surface of the seal tip. The trocar assembly may further comprise a zero closure seal such as a double duckbill valve disposed in the channel outside of the septum seal. The septum shield operates to reduce the drag force and to minimize axial movement of the septum shield and the instrument during insertion and removal of the instrument through the septum seal. The septum shield further operates to center and guide the surgical instrument through the blades or leaflets before expanding the orifice of the septum seal. The septum shield may be formed from a rigid plastic material, and the septum seal may be formed of an elastomeric material including a low durometer polymer. The blades or leaflets of the septum shield may overlap or offset one another.

In another aspect of the invention, the septum shield may be placed inside the septum seal such that the blades or leaflets engage the septum seal radially of the portions defining the orifice. The orifice may be expandable to accommodate the instrument having a diameter of about 5 mm to about 15 mm. Each of the blades or leaflets has a distal tip that glides or rolls against the instrument during insertion and removal of the instrument. In another aspect, the distal tip of each of the blades may further comprise a first material having a first durometer and a second material distal to the first material having a second durometer. With this aspect, the first durometer may be greater than or equal to the second durometer, or the first durometer may be less than the second durometer. In another aspect, the trocar assembly may further comprise a second septum shield disposed outside of the septum seal.

By locating the blades or leaflets of the septum shield near the septum seal, the drag force required to insert or remove the instrument can be reduced by allowing the instrument to slide on a lubricious material of the septum shield rather than the soft, flexible material of the septum seal. In addition, the septum shield can be used to support the septum seal and reduce any axial movement of the septum seal as the instrument is introduced or removed from the septum seal. Moreover, the blades or leaflets may be offset from each other so that as the instrument is inserted or removed, the blades or leaflets will not hang up on each other and will overlap each other repeatedly in the same fashion.

In another embodiment of the invention, a valve assembly is disclosed that is adapted to receive a surgical instrument having a cross-sectional dimension, the valve assembly comprising a housing defining a channel extending therethrough along an elongate axis; a septum seal disposed in the housing having distal portions defining an orifice, the distal portions being expandable radially outwardly to enlarge the orifice; and a septum shield operably attached to the housing and engaging the septum seal outwardly of the orifice for enlarging the orifice, the septum shield being responsive to the instrument for enlarging the orifice in proportion to the cross-sectional dimension of the instrument, the septum shield including a proximal end and a distal end, the distal end comprising a plurality of blades, each of the blades having a distal end being adapted to engage the septum seal radially of the distal portions defining the orifice.

In yet another embodiment of the invention, a seal assembly is disclosed that is adapted to receive an elongate object and to form a seal around the object, the seal assembly comprising a housing defining a channel configured to receive the object moving generally axially through the channel; a septum extending across the channel of the housing; portions of the septum defining a hole adapted to receive the object with the septum portions engaging the object through the channel; and a septum shield placed within the housing having a proximal end a distal end, the distal end comprising a plurality of blades that facilitate guidance of the object toward the hole and enlargement of the hole in response to insertion of the object into the channel.

In yet another aspect of the invention, the material, durometer and shield geometry of the blades or leaflets may be modified to control the behavior of the septum shield as an instrument comes into contact with the septum shield. It is further contemplated that the septum shield would work to open and protect the septum seal during insertion and removal of an instrument yet deflect away from the instrument as the instrument is removed in order to avoid collapsing the shield, septum seal and shield inversion phenomena, and causing a lockup or jam as the instrument is removed from the trocar.

These and other features of the invention will become more apparent with a discussion of the various embodiments in reference to the associated drawings.

### DESCRIPTION OF THE DRAWINGS

The accompanying drawings, which are included in and constitute a part of this specification, illustrate the embodiments of the invention and, together with the description, explain the features, advantages and principles of the invention. In the drawings:

FIG. 1 is a side elevation view of a prior art trocar partially cut away to illustrate a zero closure valve;

FIG. 2 is a perspective view of a prior art trocar seal including a seal sleeve;

FIG. 3 is an enlarged cross-sectional view of a septum shield of the invention replacing the seal sleeve in FIG. 2;

FIG. 4 is an enlarged cross-sectional view of a trocar seal including the septum shield of the invention;

FIG. 5 is an enlarged side view of the septum shield of the invention;

FIG. 6 is a bottom view of the blades or leaflets of the septum shield of the invention;

FIG. 7 is an enlarged side view of a septum shield in accordance with another embodiment of the invention;

FIG. 8 is a perspective view of a septum shield in accordance with another embodiment of the invention; and

FIGS. 9a-9c illustrate additional embodiments of the shield geometry of the invention.

### DESCRIPTION OF THE INVENTION

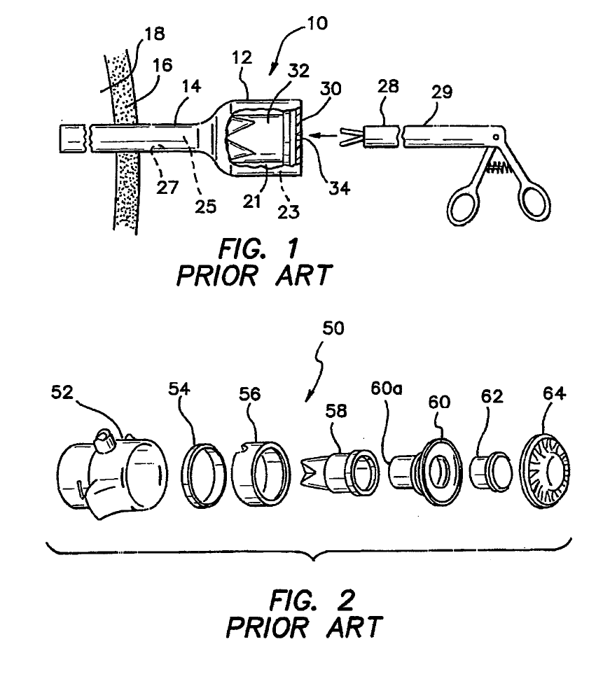

A trocar of the prior art is illustrated in FIG. 1 and designated generally by the reference numeral 10. The trocar 10 is representative of many types of surgical access devices which include a housing 12 and a cannula 14 which is adapted to extend across a body wall 16 into a body cavity 18. In the case of the trocar 10, the cannula 14 is configured to extend through an abdominal wall 16 into a cavity, such as the abdominal cavity 18. The housing 12 includes a chamber 21 which is defined by an inner surface 23. This chamber 21 of the housing 12 communicates with a lumen 25 in the cannula 14 which is defined by an inner surface 27.

The trocar 10 is commonly used in laparoscopic surgery wherein the abdominal cavity 18 is pressurized with an insufflation gas in order to provide for organ separation and otherwise increase the size of the operative environment. With these features, the trocar 10 is adapted to receive an instrument 28 having an elongate configuration and an outer surface 29. The instrument 28 is illustrated to be a pair of scissors having a length sufficient to pass through the trocar 10 and into the cavity 18 to perform a surgical operation. Although scissors are illustrated in FIG. 1, it will be understood that the instrument 28 may include any variety of devices such as needles, retractors, scalpels, clamps and various other surgical devices.

The housing 12 is configured to provide structural support for a seal mechanism, which includes an aperture or septum seal 30 and a zero closure seal 32. It is the function of these seals 30, 32 to prevent the escape of any pressurized fluid from the cavity 18 whether the instrument 28 is operatively disposed in the trocar 10 or whether the instrument 28 is removed from the trocar 10. In either case, it is desirable that the valves 30, 32 be configured to produce minimal friction forces as the instrument 28 is inserted into and removed from the trocar 10. Currently, the valve 30 will typically be formed of an elastomeric material so that the aperture 34 is biased to seal against the outer surface 29. In order to avoid significant friction forces, this aperture 34 is preferably sized to a diameter slightly less than the diameter of the surface 29. However, since various instruments and various diameters for the outer surface 29 may be required in a particular surgery, the valve 30 may have to be changed in order to accommodate a range of instrument sizes.

Referring to FIG. 2, there is shown a trocar seal 50 that is also presently being used. Trocar seal 50 comprises a seal housing 52, a cannula seal 54, a seal spacer 56, a double duckbill valve 58, a septum seal 60, a seal sleeve 62, and a seal cap 64. A drawback of the trocar seal 50, however, is that the seal sleeve 62 does not provide protection to tip 60a of the septum seal 60 during insertion of surgical instruments. Moreover, the seal sleeve 62 does not sufficiently reduce the drag force encountered when placing or removing instruments through the septum seal 60, and restrict seal movement in the axial direction of the seal and surgical instruments.

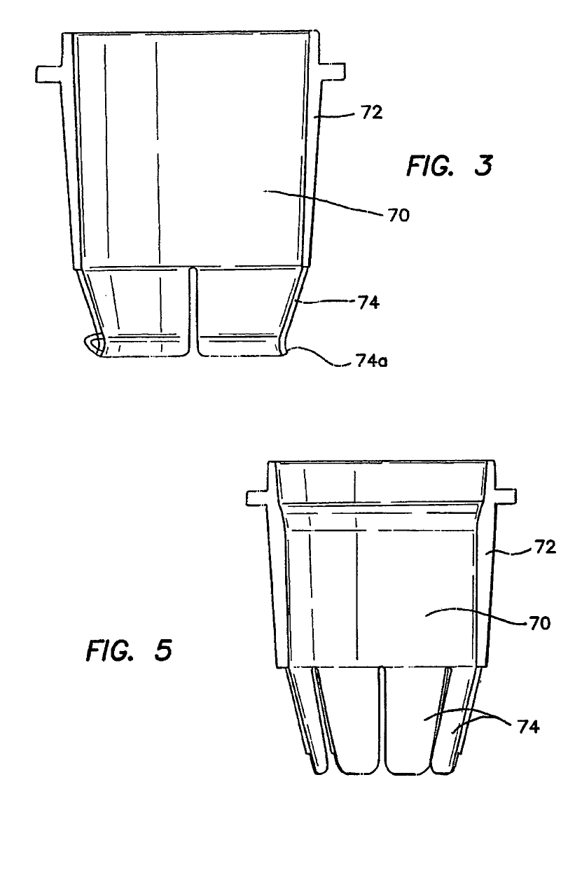

FIG. 3 illustrates a septum shield 70 of the invention that may be used in place of the seal sleeve 62 in FIG. 2 to protect septum seal 60 during the insertion and removal of surgical instruments. As explained above, the septum seal 60 operates to retain pneumoperitoneum while an instrument is placed through the respective trocar seal. The septum shield 70 of the invention includes a tubular member 72 having a proximal end and a distal end, and a plurality of blades or leaflets 74 protruding from the distal end of the tubular member 72. The septum shield 70 is placed inside the septum seal 60 such that the blades or leaflets 74 cover the top or proximal surface of the septum seal 60, including tip 60a.

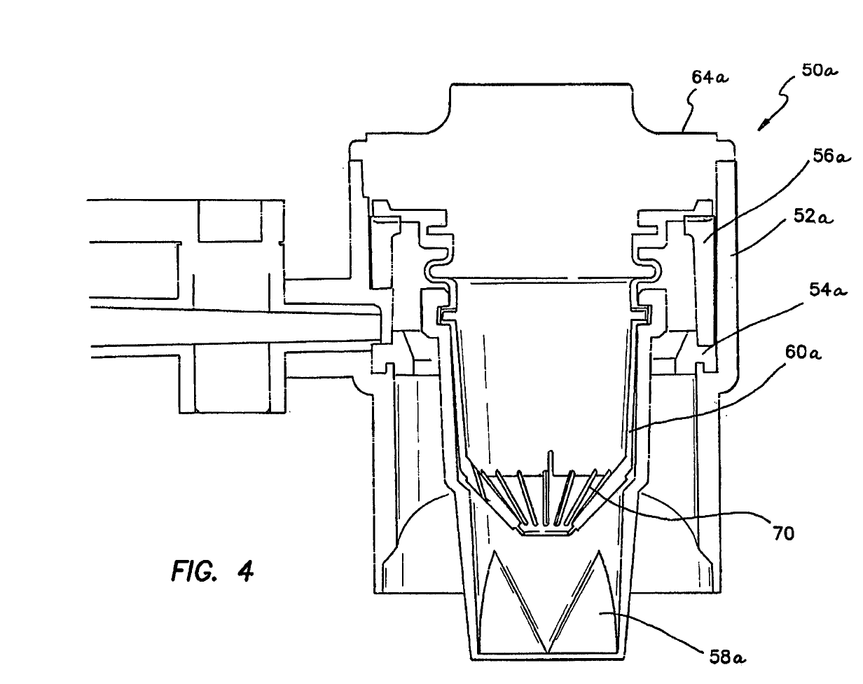

A feature of the septum shield 70 is it protects the seal 60 during the insertion of surgical instruments, it reduces the drag force encountered when placing or removing instruments through the seal 60, it restricts septum seal movement in the axial direction of the seal and surgical instruments, and it is easy to manufacture. Similarly to the trocar seal 50 of FIG. 2, FIG. 4 illustrates the interior structure of a trocar seal 50a of the invention which comprises a seal housing 52a, a cannula seal 54a, a seal spacer 56a, a double duckbill valve 58a, a septum seal 60a, a septum shield 70, and a seal cap 64a. The double duckbill valve 58a operates to provide zero seal when no instruments have been located through the trocar seal.

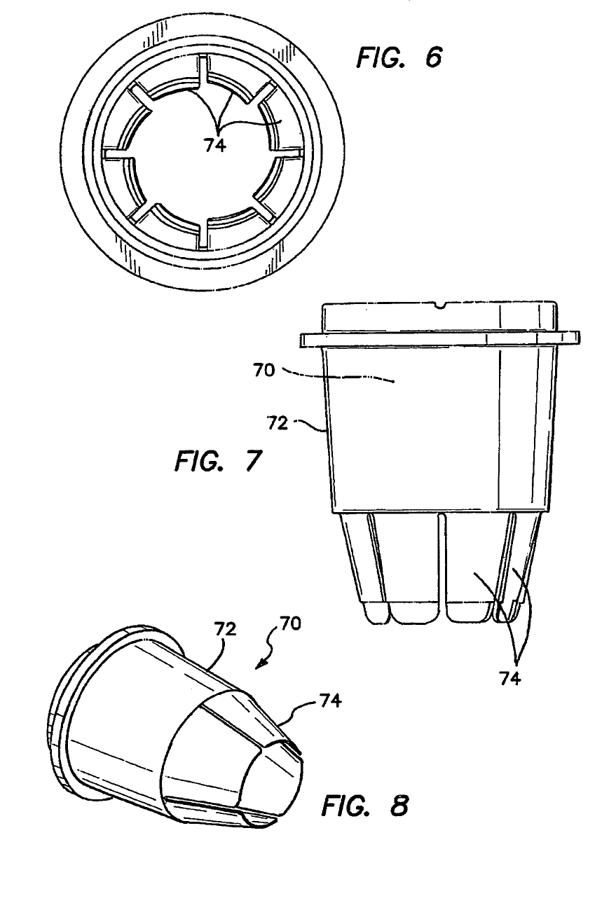

The septum seal 60a is preferably made of a soft, flexible material with an opening that expands to seal instruments ranging from about 5 mm to about 15 mm in diameter. Located within the cylinders of both the double duckbill valve 58a and the septum seal 60a is the septum shield 70 of the invention. The septum shield 70 may be formed of a rigid plastic cylinder, which operates to center and guide an instrument as it is inserted through the trocar seal 50a to the septum seal 60a. An advantage of the septum shield 70 of the invention over the sleeve 62 as shown in FIG. 2 is that the new design includes a plurality of blades or leaflets 74 that are molded into the rigid cylinder or tubular member 72. The blades or leaflets 74 protrude distally of the cylinder or tubular member 72 to cover the top or proximal surface of the septum seal 60a, including the tip 60a. FIGS. 5-8 illustrate additional views and embodiments of the septum shield 70 of the invention.

In another aspect of the invention, the blades or leaflets 74 may overlap one another and cover the proximal surface of the septum seal 60a. As the septum expands to accommodate instruments ranging from about 5 mm to about 15 mm, the respective blades or leaflets 74 work to open or expand the septum seal 60a and protect the soft, flexible material of the septum from damage due to the surgical instruments. In order to avoid potential “lock-up” as instruments are removed, the septum shield 70 of the invention is designed to deform at the tip 74a of each blade or leaflet such that each individual leaflet's tip will roll inward toward the instrument and create a variable radius for the instrument to glide on as it is removed from the seal. As the tip 74a of each leaflet is deformed inward, the body or proximal portion of the blades or leaflets 74 are forced away from the axial position of the instrument. This characteristic is accomplished by providing two stress concentrations within the design of each leaflet. The distal stress concentration allows the tip of the leaflet to move inward while creating a moment to cause the body of the leaflet to move outward and away from the instrument. The proximal stress concentration allows each leaflet to move independently from each other and the support structure of the cylinder that each leaflet attaches to.

In previous designs as illustrated in FIGS. 1 and 2, the shields such as sleeve 62 had been mounted perpendicular to the instrument movement. In the invention, the blades or leaflets 74 as illustrated in FIG. 3 are located within the conical shape of the septum seal 60a. By locating the blades or leaflets 74 of the shield near the septum, the drag force required to insert or remove instruments can be reduced by allowing the instruments to slide on a lubricious material of the shield rather then the soft, flexible material of the actual septum. This is a significant advantage over the shields of the prior art. In addition, the shield 70 of the invention can be used to support the septum and reduce any axial movement of the septum as instruments are introduced or removed from the seal 60a. Furthermore, the blades or leaflets 74 have been offset from each other and are not circumferential so that as the shield 70 is installed during manufacturing or after a large instrument has been removed, the blades or leaflets 74 will not hang up on each other and will overlap each other repeatedly in the same fashion.

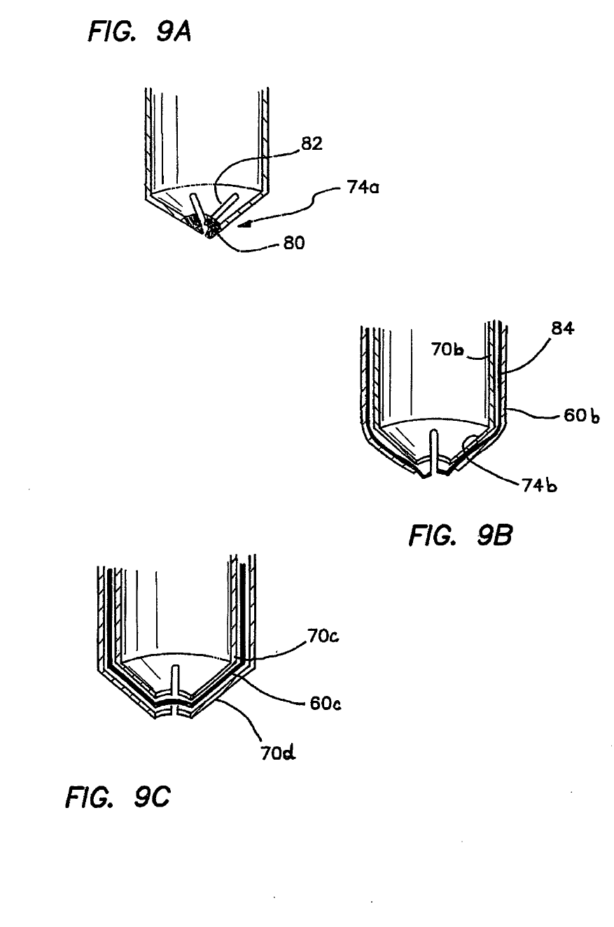

In another aspect of the invention, the material, durometer and shield geometry of the blades or leaflets may be modified to control the behavior of the shield as instruments and tools come into contact with it. For example, referring to FIG. 9a, the tip 74a may comprise of a first material 80 having a first durometer and a second material having a second durometer. The first durometer may be greater than, equal to or less than the second durometer. In another aspect of the invention as illustrated in FIG. 9b, another layer 84 may be sandwiched between the septum seal 60b and the shield 70b, the layer 84 may be formed of the same material as the shield 70b and may extend longer than the blades or leaflets 74b. In yet another aspect of the invention as illustrated in FIG. 9c, a shield 70d is provided outside the septum seal 60c. It is contemplated that the durometer or stiffness of inner shield 70c may be greater than, equal to or less than outer shield 70d, and that inner shield 70c may be shorter in length than outer shield 70d. It is further contemplated that the shields would work to open and protect the septum seal during insertion and removal of an instrument yet deflect away from the instrument as the tool is removed in order to avoid collapsing the shields, septum and shield inversion phenomena, and causing a lockup or jam as an instrument is removed from the trocar.

Many alterations and modifications may be made by those having ordinary skill in the art without departing from the spirit and scope of the invention. For example, it is contemplated that the geometry, material, and placement of the blades or leaflets and shield may be modified for different applications. Therefore, it must be understood that the illustrated embodiments have been set forth only for the purposes of examples and that they should not be taken as limiting the invention.

1. A trocar assembly having a channel defined along an elongate axis, the trocar assembly being adapted to receive a surgical instrument, the trocar assembly comprising:
a septum seal disposed in the channel including a seal tip having a proximal facing surface, the seal tip including portions defining an orifice; and
a septum shield including a tubular member having a proximal end and a distal end, and a plurality of blades protruding distally from the distal end of the tubular member, the septum shield being placed inside the septum seal such that the blades engage the proximal facing surface of the seal tip.

2. The trocar assembly of claim 1, further comprising a zero closure seal disposed in the channel outside of the septum seal.

3. The trocar assembly of claim 2, wherein the zero closure seal is a double duckbill valve.

4. The trocar assembly of claim 1, wherein the septum shield reduces the drag force during insertion and removal of the surgical instrument through the septum seal.

5. The trocar assembly of claim 1, wherein the septum shield operates to minimize axial movement of the septum shield and the surgical instrument during insertion and removal of the instrument.

6. The trocar assembly of claim 1, wherein the septum shield is formed from a rigid plastic material.

7. The trocar assembly of claim 1, wherein the blades overlap one another.

8. The trocar assembly of claim 1, wherein the septum shield is placed inside the septum seal such that the blades engage the septum seal radially of the portions defining the orifice.

9. The trocar assembly of claim 1, wherein the orifice is expandable to accommodate the surgical instrument having a diameter of about 5 mm to about 15 mm.

10. The trocar assembly of claim 1, wherein each of the blades has a distal tip that glides or rolls against the surgical instrument during insertion and removal of the instrument.

11. The trocar assembly of claim 10, wherein each of the blades provides a first stress concentration at the distal tip and a second stress concentration along the body of the blade.

12. The trocar assembly of claim 11, wherein the first stress concentration allows the distal tip to move inward against the instrument and the second stress concentration allows the body to move outward and away from the instrument.

13. The trocar assembly of claim 1, wherein the blades are offset to one another.

14. The trocar assembly of claim 1, wherein the septum seal is formed of an elastomeric material including a low durometer polymer.

15. The trocar assembly of claim 1, wherein the septum shield operates to center and guide the surgical instrument through the blades before expanding the orifice of the septum seal.

16. The trocar assembly of claim 10, wherein the distal tip of each of the blades comprises a first material having a first durometer and a second material distal to the first material having a second durometer.

17. The trocar assembly of claim 16, wherein the first durometer is greater than or equal to the second durometer.

18. The trocar assembly of claim 16, wherein the first durometer is less than the second durometer.

19. The trocar assembly of claim 1, further comprising a second septum shield being disposed outside of the septum seal.

20. The trocar assembly of claim 19, wherein the septum shield has a first durometer and the second septum shield has a second durometer.

21. The trocar assembly of claim 20, wherein the first durometer is greater than or equal to the second durometer.

22. A valve assembly adapted to receive a surgical instrument having a cross-sectional dimension, comprising:
a housing defining a channel extending therethrough along an elongate axis;
a septum seal disposed in the housing having distal portions defining an orifice, the distal portions being expandable radially outwardly to enlarge the orifice; and
a septum shield operably attached to the housing and engaging the septum seal outwardly of the orifice for enlarging the orifice, the septum shield being responsive to the instrument for enlarging the orifice in proportion to the cross-sectional dimension of the instrument,
the septum shield including a proximal end and a distal end, the distal end comprising a plurality of blades, each of the blades having a distal end being adapted to engage the septum seal radially of the distal portions defining the orifice.

23. The valve assembly of claim 22, further comprising a zero closure seal disposed in the channel outside of the septum seal.

24. The valve assembly of claim 22, wherein the septum shield is formed from a rigid plastic material.

25. The valve assembly of claim 22, wherein the orifice is expandable to accommodate the surgical instrument having a diameter of about 5 mm to about 12 mm.

26. The valve assembly of claim 22, wherein the blades are offset to one another.

27. The valve assembly of claim 22, wherein the septum seal is formed of an elastomeric material including a low durometer polymer.

28. The valve assembly of claim 22, wherein the septum shield operates to center and guide the surgical instrument through the blades before enlarging the orifice of the septum seal.

29. The valve assembly of claim 22, further comprising a second septum shield being disposed outside of the septum seal.

30. A seal assembly adapted to receive an elongate object and to form a seal around the object, comprising:
a housing defining a channel configured to receive the object moving generally axially through the channel;
a septum extending across the channel of the housing;
portions of the septum defining a hole adapted to receive the object with the septum portions engaging the object through the channel; and
a septum shield placed within the housing having a proximal end a distal end, the distal end comprising a plurality of blades that facilitate guidance of the object toward the hole and enlargement of the hole in response to insertion of the object into the channel.

31. The seal assembly of claim 30, wherein the blades of the septum shield reduce friction forces to facilitate further movement of the object though the channel of the housing.

32. The seal assembly of claim 30, further comprising a zero closure seal disposed in the channel outside of the septum.

33. The seal assembly of claim 30, wherein the septum shield is formed from a rigid plastic material.

34. The seal assembly of claim 30, wherein the hole is expandable to accommodate the object having a diameter of about 5 mm to about 15 mm.

35. The seal assembly of claim 30, wherein the blades are offset to one another.

36. The seal assembly of claim 30, wherein the seal is formed of an elastomeric material including a low durometer polymer.

37. The seal assembly of claim 30, further comprising a second septum shield being disposed outside of the septum.
